(19) United States
(12) Patent Application Publication
(10) Pub. No.: US 2010/0120014 A1
(43) Pub. Date: May 13, 2010

(54) Stability Drying

(76) Inventor: Victor Bronshtein, San Diego, CA (US)

Correspondence Address: Victor Bronshtein, Ph.D., 5008 Almondwood Way, San Diego, CA, 92130, US

(21) Appl. No.: 12/462,855

(22) Filed: Aug. 10, 2009

Related U.S. Application Data
(63) Continuation-in-part of application No. 10/174,007, filed on Jun. 18, 2002, now abandoned.

(51) Int. Cl. C12N 9/96; C12N 1/04; C12N 5/02; A01N 1/00

(52) U.S. Cl. 435/1.1; 435/188; 435/260; 435/374; 435/420

(57) ABSTRACT

A method of formulating high ambient temperature (room temperature and above) stable biologics (biologically active macromolecules, enzymes, serums, vaccines, viruses, pesticides, drug delivery systems, liposomes, cells suspensions, sperm, erythrocytes, other blood cells, stem cells, multicellular tissues, skin, heart valves) including secondary drying comprising at least two steps of stability drying at elevated temperature: 35° C., 40° C., 45° C., 50° C., and higher temperatures. The method could be applied to stabilize biologics encapsulated in alginate gel microspheres for better oral delivery. The method encompasses the following: microspheres are formulated using a cryo-encapsulation procedure comprised of mixing drops of frozen preservation mixture (To form the preservation mixture, biologics are mixed with preservation solutions containing sodium alginate.) with frozen drops of a calcium solution (i.e. calcium gluconate) and subsequent warming to form the gel particles.

### CONTINUING APPLICATION INFORMATION

This application is a continuation-in-part and claims the benefit of priority of U.S. utility patent application Ser. No. 10/174,007, filed Jun. 18, 2002, titled “Long-Term Shelf Preservation by Vitrification”, which in turn claims the benefit of priority of U.S. provisional patent application Ser. No. 60/018,573, filed May 29, 1996.

### FIELD OF THE INVENTION

The invention relates to methods for long-term high temperature (room and higher temperatures) preservation (stabilization) of the activity of biologics: biologically active molecules (therapeutic proteins, enzymes, factors, etc.), viruses, bacteria and other cells, small (or thin) multicellular complexes (i.e. embryos, skin, etc). Specifically this invention is related to the field in which this stabilization is achieved by immobilizing the biologics in the dehydrated glasses comprising protective carbohydrates.

### BACKGROUND OF THE INVENTION

The long-term storage of biologically active materials, including but not limited to cells and multicellular tissues, is becoming more and more necessary for both commercial and research purposes, yet such materials may be the most difficult to store of any materials known. Ironically, the same properties which make biologically active agents and life forms valuable are the properties which make them so difficult to preserve. Certainly very few such materials are sufficiently stable to allow them to be isolated, purified and stored in room temperature solution for anything more than a very short period of time.

Both commercially and practically, shelf storage of dehydrated, biologically active materials carries, with it, enormous benefits. Successfully dehydrated reagents, materials, and cells have reduced weight and require reduced space for storage, notwithstanding their increased shelf life. Room temperature storage of dried materials is, moreover, cost effective when compared to low temperature storage options and their concomitant costs. The biologically active materials addressed herein include but are not limited to biologically active macromolecules, enzymes, serums, vaccines, viruses, pesticides, drug delivery systems, liposomes, cells suspensions, sperm, erythrocytes, other blood cells, stem cells, multicellular tissues, skin, heart valves, and so on.

As the benefits of shelf preservation of biological specimens has become more appreciated, researches have endeavored to harness vitrification technology in the biological world. Vitrification refers to the transformation of a supercooled liquid into an amorphous solid glass, which occurs upon rapid cooling, which occurs if crystallized phase nucleation and growth have not had enough time to happen before the glass is formed. Thus, vitrification is a non-equilibrium dynamic phenomenon occurring between two distinct states of matter (liquid and crystallized solid state), each with different physical properties.

While liquid-to-glass transition may not yet be completely understood, it is well established that liquid-to-glass transition is characterized by a simultaneous decrease in entropy, sharp decreases in specific heat and expansion coefficient, and tremendous (more than thousands of millions times) increases in viscosity with corresponding decrease in mobility and diffusion rates. Several microscopic models have been proposed to explain liquid-to-glass transition, including free volume theory, percolation theory, mode coupling theories and others. Theories are unimportant, however, as long as, in the practice of the invention, reliable experimental methods for establishing the glass transition temperature (T_{g}) are used. The recommended methods are: (a) measurement of the change in specific heat and (b) measurement of temperature stimulated polarization (TSP) or depolarization currents described in the art.

We found in our research that onset temperature of the change in specific heat (Cp) occurred during warming of sugar and other carbohydrate glasses occurred at the same temperature as the onset of TSP (that reflects the beginning of the mobility of the glass specimens during warming). For this reason we believe that measuring onsets of Cp and TSP provides a more reliable estimation of T_{g}.

The technology of vitrifying or achieving the glass state for any given material has been anticipated to emerge as a premier preservation technique for the future, although prior art vitrification techniques have been plagued with unexpected problems. As the developments underlying the invention will illustrate, although Applicant does not intend to be bound by this theory, in retrospect it would appear that fear of sample damage has inhibited previous investigators from considering an appropriate temperatures for dehydration in order to truly achieve the glass state of any given material at room or higher ambient temperature. As a result, previous attempts at preservation have generally yielded inferior products, with excessive water content or having properties inconsistent with a true glass state. These products generally exhibit limited storage stability at room or higher temperature. We are not saying that being in a true glass state 100% ensures stabilization of activity of biologics immobilized in the glass state. Still there could be some processes running inside the lipid phase of the membrane phase or inside protein globules separated from immobile sugar glass environment. However, we are saying that at temperatures above T_{g}, mobility in the liquid phase will be characterized by diffusion limited processes (including denaturation of proteins, chemical reactions between active groups and molecules, etc.) that will result in a continuous decrease in activity. Thus, being in the true glass state where the mobility is arrested is required for long-term stabilization of biologics.

An important misconception has endured due to the belief that vitrification can be achieved by drying alone. References are numerous in which substances are purported to have achieved a true glass state by drying, yet the disclosed techniques do not actually result in a glass state forming. The true statement is that because drying is a process, limited by diffusion of water molecules, the glass state, at constant hydrostatic pressure, can be achieved only by cooling. It should be noted that, prior to our disclosure of this simple physical idea in our provisional patent application Ser. No. 60/018,573, filed May 29, 1996, this fact was not appreciated. In this context, it is important to note issued patents in which this misconception is misleadingly embodied.

Wettlaufer and Leopold, U.S. Pat. No. 5,290,765, patented a method of protecting biological materials from destructive reactions in the dry state. They suggest protecting the biological suspension during drying and subsequent storage by combining the suspensions with sufficient quantities of one or more vitrifying solutes and recommended a 3/1 weight percent sucrose/raffinose mixture. The materials are taught as intended to be dried until drying is insufficient, but this is misleading and an erroneous teaching. At best, these materials achieve a very viscous liquid state, which resembles a rubbery state, but no glass state ever emerges.

Franks et al., in U.S. Pat. No. 5,098,893, likewise teaches that all that is necessary to achieve the glass state at ambient temperature is evaporation at ambient temperature and that any optional temperature increase should be imposed only to increase evaporation rate. For this reason, even though Franks et al. believed that the samples described in their examples achieved the glass state, in actuality they did not.

The misconception explained above has occurred for several reasons. First, some individuals have used the terms “glass”, “glassy”, and “vitrified” in a vague and hence misleading way. Second, it is admittedly difficult to measure reliably the glass transition temperature (T_{g}) of dry mixtures containing polymers or biopolymers. The change in specific heat in such mixtures is very small and occurs over a broad temperature range that makes reliable differential scanning calorimetry (DSC) measurements of T_{g} difficult. When the measurement is omitted, certain individuals assume that a glass state has been achieved when it has not. Third, sometimes more water remains in a supposedly vitrified material than would be consistent with a true glass state, but in many cases measurement of this water for a variety of reasons gives an erroneous result. All of these reasons, and probably others, tend to fuel the wishful thinking that a glass state has been achieved when it has not. Because the diffusion coefficient of water quickly increases with increasing temperature above the glass transition temperature, with prior art preservation methods. the safe storage time is limited if samples are stored above the glass transition temperature.

A need thus remains for a preservation protocol which effects true vitrification and long-term stability of biologically active materials at high (room, 37° C., and higher) temperatures, including but not limited to peptides, proteins, other molecules and macromolecules, and cells.

### SUMMARY OF THE INVENTION

One embodiment of the present invention is a method of shelf preserving biologically active specimens by vitrifying them, i.e. formulating them in such a way as to achieve a true glass state. In one embodiment, the method is founded that to store samples in a true glass state and achieve long-term stability, the dehydration (drying) temperature of the material must be higher than the suggested storage temperature (Tₛ). For example, implementing this embodiment of the present invention in some cases requires drying (preferably under vacuum) at 40° C., 50° C., or even 60° C. or above temperatures, followed by subsequent cooling to high ambient (room, 37° C., and higher storage temperatures) storage temperatures. Drying at the elevated temperatures (40° C., 50° C., or even 60° C.) could be very damaging. Surprisingly, we found that protection from the damaging effect of the elevated temperatures could be achieved by dehydration (drying) that, if not executed properly, could be very damaging to the biologics we intended to preserve.

Dehydration is not a direct damaging factor to a viral or other biological structure and function. Most of the dehydration-induced damage to unprotected biologics is associated with hydration forces that rise between biological membranes and macromolecules when the distance between them becomes very small. This dehydration-induced damage can be diminished by replacing a portion of the water of hydration before drying with protective carbohydrates (fillers) that replace the water and adsorb to the surface of the biological membranes and macromolecules. The fillers protect in two ways: they eliminate hydration forces and they also create glassy shells around the biological membranes or macromolecules. Thus, in the presence of protective fillers, drying is a protective phenomenon because it protects biologics from irreversible damage that could happen at elevated temperatures.

Because of that, in other instances, to avoid or minimize the damage of a biologic to be preserved the present method requires careful stepwise (or continuous) elevation of the drying temperature with simultaneous dehydration of the specimen preferably under vacuum. The drying temperature should not be elevated to the next level until protective level of dehydration is reached. Too quick elevation of the drying temperature could result in irreversible loss of the activity of the specimen. At the same time too slow elevation of the drying temperature also could result in the loss of activity because the specimen actual T_{g} is always below the drying temperature and the material could be only stable during a short-times at this conditions.

Because activity of wet biological materials will be quickly damaged at elevated temperatures (above RT), the drying at these temperatures can be performed only after the primary drying of the material is complete. Primary drying can be performed by freeze-drying, spray drying, preservation by foam formation, preservation by vaporization, belt drying, dram drying and other scalable drying methods that allow removing about 90-95% of the water from the materials without strongly damaging the activity of the material. We also will call the primary drying as the first drying step.

Typically, after primary drying, the material is stable only at 0° C. or below. Secondary drying should be applied to remove more water from the specimens after the primary drying by evaporation, which includes diffusion limited step of water transport from the inside of the dry material to its surface. According to this invention, it can be done only if the drying temperature is higher than T_{g}. An important embodiment of this invention is that secondary drying should include two steps of stability drying at elevated temperatures (above RT) so that the dehydration during the first step of stability drying will protect the material activity during the second step of stability drying at higher temperature.

Another embodiment of the present invention is related to high ambient temperature stabilization of biologics encapsulated in gel microspheres (particles) for better oral delivery and protection of the material from gastric juice in stomach and bile in duodenum. This case is of specific importance because removing water from inside of the microspheres during secondary drying is very difficult because the gel particles surface to volume ratio is much smaller than that for materials after freeze-drying, spray drying, foam drying, etc. Removal of the water from the microspheres during secondary drying occurs by evaporation, which is limited by diffusion of the water from the middle of a microsphere to its surface. It is well known that characteristic time (t) of the diffusion relaxation in the drop with diameter (d) is about t=d²/D, where D is the water diffusion coefficient and d is the drop diameter. In dilute solutions, D=¹⁰⁻⁵Sm²/sec and t=10 sec for small drops with diameter d=100μ. However, in drops containing concentrated solutions (syrups), t will greatly increase with a decrease of molecular mobility and diffusion coefficient (D). In concentrated syrups, D is larger than 10⁻⁵sm²/sec by many orders of magnitude, which makes t many orders of magnitude higher than the typical secondary drying process time. The solution to slow drying is increasing the drying temperature, which could damage the biological activity of the specimen. For this reason, it is very difficult (but not impossible) to achieve both good initial yield and stability of vaccines or other biologics at room, 37° C. or higher temperatures. During optimization of stability drying one should find a compromise between achieving our desired stability and activity after drying.

### DETAILED DESCRIPTION OF THE INVENTION

The present invention overcomes the deficiencies of the prior art and allows preservation and storage of specimens in the actual glass state without loss of biological activity during storage. Biological specimens, which can be vitrified to a glass state include but are not limited to proteins, enzymes, serums, vaccines, viruses, liposomes, cells, and certain multicellular specimens. The key to genuine vitrification is to conduct the dehydration at a temperature higher than the suggested storage temperature (Tₛ) to achieve the glass transition temperature (T_{g}, where T_{g}>Tₛ, followed by cooling of the sample to the suggested storage temperature Tₛ This is very difficult to achieve if the targeted storage temperature is above room temperature (RT) because the preservation process should include drying at elevated temperatures that are much higher than RT followed by cooling to room temperature.

To improve quality and prolong shelf life at storage temperatures, the samples should be dehydrated so that T_{g} actually becomes higher than Tₛ. However, because dehydration of the glassy materials is practically impossible, the only way to achieve T_{g}>Tₛ at constant hydrostatic pressure is to dehydrate the samples at a temperature that is higher than the glass transition temperature. This has to be done despite risk of heat degradation of the specimen.

Dehydration of biological specimens at elevated temperatures may be very damaging if the temperatures used are higher than the applicable protein denaturation temperature. To protect the samples from the damage associated with elevation of temperature, the dehydration process should be performed in steps. The first or primary step of the dehydration (air or vacuum) should be performed at such low temperatures that the sample can be dehydrated without loss of its activity. If the first or primary step requires dehydration at sub-zero temperatures one may apply freeze-drying techniques. After the first drying step, the dehydration may be continued by drying at higher temperatures. Each drying step will allow simultaneous increases in the extent of dehydration and temperature of drying. For example, in the case of enzyme preservation it was shown that after drying at room temperature the drying temperature may be increased to at least 50° C. without loss of enzymatic activity. The extent of dehydration obtained after drying at 50° C. will allow a further increase in the drying temperature, without loss of activity. For any given specimen to be preserved, the identity of the specimen will determine the maximum temperature it can withstand during the preservation process, i.e. denaturation temperature, etc. It should be noted, however, that various protectants and cryoprotectants confer protection to materials to be dried during the drying process, i.e., sugars, polyols and polymeric cryoprotectants.

It should also be noted that, according to one embodiment of the invention, all methods of successful freeze-drying and drying of biological materials and specimens reported so far can be optimized by the additional stability drying with subsequent cooling to vitrify the specimen. The vitrified samples can then be stored on a shelf for long-terms.

One embodiment of the present invention is a method of shelf preservation of biological specimens by true vitrification comprising dehydrating a biologically active material at a temperature higher than the suggested storage temperature, followed by cooling of the sample to the storage temperature.

Another embodiment of the present invention is a method of shelf preservation of a biologically active material containing a protectant by true vitrification at room or higher temperatures, comprising treating the biologically active material by:

(a) drying said biologically active material in a first drying step, wherein said step is selected from the group consisting of evaporation under vacuum, freeze-drying, spray-drying, or belt-drying methods used to reduce a major quantity of solvent in the biologically active material; and
(b) continuing to dry said biologically active material in at least two additional drying steps at elevated temperatures with increasing temperature during each subsequent step, wherein the drying temperature during the last drying step is higher than targeted storage temperature, and with said last drying step continuing for a period of time sufficient to increase the glass transition temperature of said biologically active material; followed by
(c) cooling said biologically active material to said storage temperature, wherein said drying and cooling steps yield a stable vitrified biologically active material.

### Example 1

### Example of Stability Drying

It is well known that most of freeze-dried vaccines are not stable at ambient temperatures. For example, we had recently reported that freeze-dried (F-D) YF-Vax 17D strain of Yellow fever virus (lot UF057AA, Sanofi Pasteur Inc.) and F-D MVA vaccine (ACAM 3000, lot 460301 DA) lose more than 1 log in activity yield after 4 months of storage at 37° C. To better stabilize the same vaccines we performed reformulation that included 20 hours stability drying at 45° C. No decrease in vaccine activity was observed after reformulation and subsequent storage for more than 4 months at 37° C. The vaccines were preserved using our PBV technology reported earlier (PCT Patent Application WO051 1 7962A). To perform PBV stabilization we reconstituted each vial containing freeze-dried vaccines with a preservation solution comprising 28% Isomalt and 12% Methylglucoside (MAG.).

### Example 2

### Preservation of Lactobacillus GG (LGG) Encapsulated in Alginate Gel

LGG is a well known probiotic bacteria that has been used as an additive to baby food (“Nutramigen-2”) to protect infants from colonization of the intestinal epithelium with pathological bacteria. LGG fermentation was performed inside a 2 L BioFlo fermentor in MRS broth +0.05% cysteine at 37° C. The culture was centrifuged, the supernatant was decanted and pellet was re-suspended 1:1 with a Preservation Solution (0.7% of alginate, 0.7% of high amylase starch, 20% of isomalt and of 10% methylglucoside) to obtain a Preservation Mixture (PM). In this experiment, to form the gel particles, we had used a conventional procedure for preparation of alginate gel microspheres:

The preservation mixture was sprayed in a bath solution containing 2% CaCl₂ and protective sugar derivatives: 10% of isomalt and of 5% methylglucoside. The bacteria in the gel particles were preserved using two PBV drying processes. The stability drying temperatures were 35° C. and 50° C. during the first and second PBV processes, respectively.

To measure bacterial survival before and after drying, 100 mg of wet particles (before drying) and 20 mg of dry particles after drying, which had been stored at RT, were reconstituted with 5 ml of 0.5% sodium hexametaphosphate (SHMP) buffer. Sodium hexametaphosphate chelate Ca++ alginate gel particles dissolve in the buffer, thus releasing the bacteria entrapped in the alginate gel. Results:
54±1% bacteria survived drying at 35° C. After 1 month at RT the survival was 45±2%, and did not change after 2 month at RT (44±3%). However, it significantly decreased to 6±1% after 2 weeks of storage at 37° C.
30±1% bacteria survived drying at 50° C. After 2 month at RT, the survival was 28±3% However, in this case, survival was not significantly decreased after 2 weeks of storage at 37° C. (27±2%).

Thus the higher the stability drying temperature the higher is stability of LGG at 37° C.

### Example 3

### Stability of Lm Vaccines Formulated by PBV

We has examined the stability of a recombinant Listeria monocytogenes (Lm) vaccine constructed by Cerus Corp, and dried using our PBV technology including 20 hours stability drying at 50° C. The viability of the preserved bacteria was evaluated by plating them on BHI agar. The results below (Table 3.1) showed that the vitrified Lm was relatively stable.

TABLE 3.1
Stability of PBV-preserved rLm vaccine at 37° C.

After stability drying at 50° C.

| | CFU/plate | Stability (%) | |
|---|---|---|---|
| Before drying | 326 ± 10 | 100% | |
| After drying | 177 ± 7 | 54% | (100%) |
| 20 days at 37° C. | 154 ± 8 | 47% | (87%) |
| 2 months at 37° C. | 158 ± 8 | 48% | (89%) |
| 350 days at 37° | 117 ± 16 | 36% | (66%) |

### Stability of Lm Vaccine Formulated for Oral Use.

We also examined the stability of Lm vaccines encapsulated in alginate microspheres and dried by PBV, designed for oral delivery. To encapsulate LMV bacteria in alginate gel microspheres, we used our crosslinking method (cryo-encapsulation) comprising following steps:
a) Spraying of a preservation mixture (PM) into liquid nitrogen (LN₂) (Cryopelletization) using a paint sprayer, which produced frozen particles with diameter of about 100-300μ.
b) Spraying 10 ml of the 10% Ca⁺⁺ crosslinking solution into the same tray with LN₂.
c) Mixing the frozen microspheres in LN₂ and separating the microspheres from the LN₂.
d) Warming the frozen mixture of microspheres to obtain a concentrated suspension alginate gel microspheres.

The advantage of the crosslinking method above of producing alginate gel microspheres (gel particles) is its scalability and small amount of liquid containing extra particles, and the very low concentration of bacteria in the liquid outside of microspheres.

PBV Drying of the concentrated alginate microspheres was performed in small 200 ml Lyoguard trays (Cups). The stability drying step was performed during 24 hours under vacuum at 40° C. to achieve a higher glass transition temperature of the dry preparation. After drying, the material was broken to microspheres by gently applying a pressure on the dry material surface using a sterile spoon which turned the material into a powder. This operation was performed in a dry room at a relative humidity (RH) of about 15% to ensure that no moisture penetrated the dry powder. Then the dry microspheres were distributed into 10 ml glass vials (0.033 g per vial) for storage at room temperature (RT). The vials were sealed using conventional rubber stoppers and aluminum seals.

We measured the following:
Concentrations of live bacteria (CFU/g) in the PMs. Concentration of live bacteria was measured by plating of the bacteria (0.1 ml per plate) on BHI agar after 10⁻⁷ dilution. Plates were incubated at 37° C. for 24 hrs before CFU were counted.
Concentration of live bacteria (CFU/g) encapsulated inside alginate gel microspheres. To release bacteria encapsulated in the alginate gel phase in the dilution solution, 0.1 g of the gel particle formulation was first dissolved in 10 ml of SHMP buffer. This 10⁻² dilution was further diluted to 10⁻⁷ for plating.
Concentration of live bacteria in the liquid remaining outside of alginate gel microparticles. To measure the concentration of bacteria outside the gel phase, the liquid was separated from the gel microspheres using a 40μ sieve. 0.1 g of the liquid was first dissolved in 10 ml of SHMP buffer to release the bacteria encapsulated in particles smaller that 40μ. This 10² dilution was further diluted to 10⁻⁴ and plated (0.1 ml) on BHI agar.
Survival of the bacterial in the dry formulation after drying and subsequent storage at RT is shown in Table 3.2 below. To measure survival of the dry bacteria, 0.033 g of the dry powder gel particle formulation was reconstituted with 10 ml of SHMP buffer. This 10⁻² dilution was further diluted to 10⁻⁷. From the 10⁻⁷ dilution 0.1 ml was plated on BHI agar plates. We used 0.033 g of dry formulation because after drying of 0.1 g of wet formulation weighed about 0.033 g.

TABLE 3.2

| Results: | Dilution | CFU/plate |
|---|---|---|
| Before spraying | 10⁻⁸ | 67 ± 8 |
| After UST-crosslinking. | 10⁻⁸ | 115 ± 6 |
| After drying | 10⁻⁸ | 52 ± 9 |
| After 30 days at RT | 10⁻⁸ | 40 ± 2 |
| After 9.5 months at RT | 10⁻⁸ | 40 ± 6 |
| In the extra gel liquid | 10⁻⁵ | 130 ± 13 |

CFU in the alginate gel microspheres after drying (52±9×10⁸) decreased to (40±6×10⁸) after storage for 9.5 months at room temperature. The alginate formulation thus retained 77% viability after long-term ambient temperature storage.

### Example 4

### Preservation of Rabies Viral Vaccines

Fresh ERA-333 live attenuated vaccine for the study was provided by the Rabies Department at CDC. Fresh VRG-AY 595-451 (VRG) live attenuated vaccine for the study was provided by Merial Ltd. (Merial). The vaccines were shipped to UST frozen on dry ice.

Before drying, the vaccine was mixed (typically in 1:100 ratios) with a preservation solution (PS) to form a preservation mixture (PM). To encapsulate vaccine viruses in alginate gel microspheres we used preservation solution (PS) comprising sodium alginate (Manugel DMB from FMC Biopolymer). The PM was sprayed in a bath solution containing calcium gluconate or CaCl₂, sucrose and methylglucoside (MAG). The spraying was performed using controlled pressure compressed gas and conventional pain spraying nozzle. The gel particles were collected by passing the suspension through a screen of 90UM USA standard sieve #170 (hole size 90 μm). Preservation by Vaporization (PBV) Drying of the concentrated alginate microspheres was performed in small (200 ml) Lyoguard trays (cups). To preserve vaccines with no gel encapsulation PMs were aliquoted in serum vials (0.5 ml/vial) and dried using our PBV method (Bronshtein, 2005). The drying was performed using Genesis and Ultra freeze-dryers (from Virtis, Inc.) modified to allow better vacuum pressure control during drying. At the end of 1 to 2 hours of primary drying during which most of the water had been vaporized, the secondary drying was performed under vacuum to increase the glass transition temperature of the dry specimens. The secondary drying included two 20-24 hours stability drying steps, first at 35° C. and second at 45° C. (or 40° C.) to ensure stability of the vaccines at 37° C. and short-term (1 hour) stability at 60° C. After primary drying, the material in the vials looked like stable dry foams. The material in Lyoguard cups looked like a dry expanded cake. The cake containing dry microspheres was turned into a powder by gently applying pressure on the dry cake surface using a sterile spoon. This operation was performed at a relative humidity (RH) of about 15% to ensure that no moisture penetrated the dry powder. Then the powder was distributed into glass vials under weight control. To measure the viral titer in dry preserved specimens, the dry gel microspheres were reconstituted with 0.5% sodium hexametaphosphate (SHMP) on saline buffer. SHMP chelates calcium ions and breaks alginate gel particles, thus releasing the viruses.

1. Titration of VRG was performed at Merial on Vero cells using an SOP approved in the Merial's QC department.

2. ERA virus titration at UST was done in 96 well plates (Falcon) on BHK-21 (baby hamster kidney) cells by direct immunofluorescence assay with FITC anti-rabies monoclonal globulin (Fujirebio Diagnostics, Inc). Count of positive cells was done 24 hours after infection under a fluorescence microscope Axiolab (Zeiss). A similar protocol was used for ERA virus titration at CDC using BRS clones of BHK21 cells. The cells were grown in Glasgow-modified MEM supplemented with 10% calf serum. UST obtained the BHK21 cell starters from Dr. Rupprecht. Results obtained in parallel titration studies at UST and CDC were in good correlation with each other, however, most of the results (including control viral titer) obtained at UST were several times lower that that obtained at CDC. According to CDC titrations, results activity of fresh ERA G333 was somewhere between 1 and 3×10⁸ FFU/ml. According to measurements performed at UST activity of fresh ERA G333 was somewhere between 7 and 11×10⁷ FFU/ml. All data presented below are adjusted for the dilution which occurred during preservation and reconstitution of the vaccine specimens.

We found that the presence of active Ca⁺⁺ ion in the vaccine viral suspension strongly decreased the viral titer in both liquid state and in dry preserved state. This phenomenon was observed for both ERA-333 rabies vaccine from CDC, and VRG rabies vaccine from Merial (see section 4.2.1 below). This phenomenon damaged the vaccine viruses which were encapsulated in alginate gel microspheres. The microspheres are necessary for better oral delivery because to form the gel, alginate has to be crosslinked with Ca⁺⁺

### 4.2 RESULTS AND CONCLUSIONS

### 4.2.1 Results

4.2.1.1 Preservation of Vaccines Non Encapsulated in a Gel.

The objective of the first study was to determine the optimum composition of protective carbohydrates that would allow stability of the vaccines at least a week at 37° C. and 1 hour at 60° C. In the study with ERA-333 we evaluated several PS formulations described in the Table 4.1 below.

TABLE 4.1
Composition of Preservation Solutions (pH = 7)

| | | | | | | Phosphate |
|---|---|---|---|---|---|---|
| | | | | | | buffer |
| | Isomalt | Sucrose | MAG | Manitol | Gelatin | 0.01M |
| PS1 | 19% | 7% | | | | 74% |
| PS2 | 19% | | 7% | | | 74% |
| PS3 | | 19% | 7% | | | 74% |
| PS4 | 19% | | | 7% | | 74% |
| PS5 | 19% | | | 7% | 0.5% | 73.5% |

We found (see Table 4.2 below) that PS3 provided the best protection for ERA-333 vaccine during drying and subsequent storage at 37° C. Activity loss after 2 weeks at 37° C. was less than 0.5 logs. The highest activity loss was observed with PS1. ERA-333 preserved with other PS2, PS4, and PS5 lost between 1 and 2 logs of activity titer after two weeks at 37° C.

TABLE 4.2

| | | Rabies virus titer (log₁₀FFU/ml) | | | |
|---|---|---|---|---|---|
| Temperature | | PM control | | | |
| of stability | | before | 0 weeks | 1 week at | 2 weeks at |
| drying | Form. | drying | at 37° C. | 37° C. | 37° C. |
| 45° C. | PS1 | 7.22 ± 0.06 | 4.82 ± 0.41 | 5.25 ± 0.93 | ≦3 |
| | PS2 | 7.78 ± 0.06 | 7.28 ± 0.2 | 6.91 ± 0.12 | 6.32 ± 0.13 |
| | PS3 | 7.44 ± 0.03 | 7.78 ± 0.11 | 7.61 ± 0.13 | 7.18 ± 0.09 |
| | PS4 | 7.48 ± 0.16 | 7.14 ± 0.11 | 6.38 ± 0.49 | 5.48 ± 0.6 |
| | PS5 | 7.75 ± 0.16 | 7.18 ± 0.07 | 6.43 ± 0.26 | 6.3 ± 0.26 |
| none | PM3, −80° C. | | | 7.65 ± 0.08 | 7.46 ± 0.13 |

We also tested activity of the vaccine preserved with PS3 after 1 hour equilibration at 60° C. No significant loss of activity was found (see Table 4.3 below).

TABLE 4.3

| Temperature | | Rabies virus titer (log₁₀FFU/ml) | | |
|---|---|---|---|---|
| of stability | | Activity | | |
| drying | Formulation | after drying | 1 h at 60° C. | 1 h at 65° C. |
| 45° C. | 3 | 7.78 ± 0.11 | 7.64 ± 0.17 | 6.92 ± 0.11 |
| none | PM3, −80° C. | | 7.79 ± 0.2 | |

Similar results were obtained with VRG vaccine. In the study with VRG, we evaluated the following PS formulations: 28% Isomalt and 12% MAG; 28% Sucrose and 12% MAG; 25.2% Sucrose, 10.8% MAG, and 4% MSG; 25.2% Isomalt, 10.8% Mannitol, and 4% MSG.

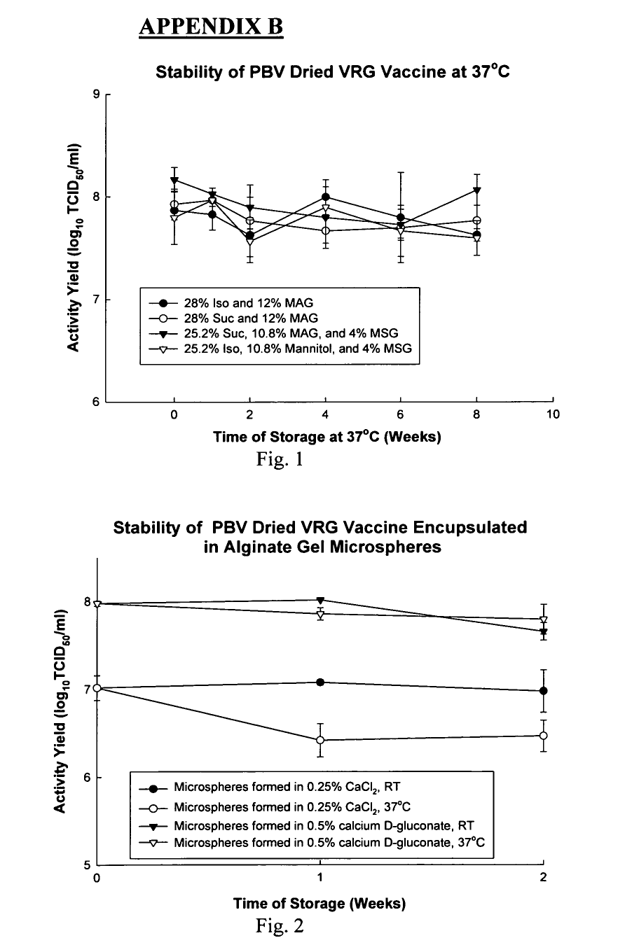

The result are shown in FIG. 1 (of two). VRG virus vaccine is stable after preservation in all PSs. We also did not detect any loss of the preserved VRG after 1 hour equilibration at 60° C. in all PS used in this experiment.

4.2.1.2 Preservation of Vaccines Encapsulated in Alginate Gel Microsphere in Calcium Chloride Solutions.

In this study we used only preservation solutions comprising sucrose and methylglucoside (MAG). As we mentioned above, we quickly found that presence of active Ca⁺⁺ ion in the vaccine viral suspension strongly decreased the viral titer in both liquid state and in dry preserved state. We observed this phenomenon first in the dry state and then evaluated it in the liquid state.

In the next experiment we used the following PSs:

PS −1:30% sucrose, 15% MAG, and
PSA −1:1% sodium alginate, 1% Monosodium glutamate (MSG), 20% sucrose, and 10% MAG.

The Preservation mixture (PMA) containing PSA was sprayed in a calcium solution (CS) containing 0.25% calcium chloride, 14% sucrose, and 7% MAG. The microspheres were collected using 90UM USA standard sieve #170 (pore size 90 mkm). The vaccine virus activity was measured in alginate particles that were collected in the solution remaining after sieving (SAS=solution after sieving), and in supernatant of SAS solution after centrifugation for 10 min at 1000 rpm (about 200 rcf) to precipitate microspheres that passed through the sieve.

### Efficacy of Encapsulation of ERA-333 Virus.

We found (Table 4.4) that the majority (97%) of the vaccine virus was encapsulated in the gel microspheres that did not pass through the sieve, reflecting high efficacy of the encapsulation procedure.

TABLE 4.4

| | ERA-333 virus titer |
|---|---|
| | (log₁₀FFU/ml) |
| PMA | 7.64 ± 0.02 |
| AP | 7.69 ± 0.09 |
| SAS | 6.09 ± 0.13 |
| SAS supernatant | 4.24 ± 0.59 |

Stability of ERA-333 Rabies vaccine after drying, encapsulated and “non-encapsulated” in alginate gel microspheres The drying process did not significantly affect the virus activity of non-encapsulated vaccine, but was damaging to the encapsulated virus, with loss of activity of about 1.9 logs FFU/ml. Similar observations were obtained with VRG vaccine (see FIG. 2 (of two). Both encapsulated and non-encapsulated rabies virus vaccines were stable at RT during an observation period of 2 weeks. Storage at 37° C. of non-encapsulated vaccine affected virus activity only during the second week, with loss of activity 0.6 logs FFU/ml; this is consistent with the results shown in Table 4.2. However encapsulated vaccine lost 0.5 logs after storage 1 week at 37° C. and lost 0.9 logs FFU/ml after the second week of storage at 37° C. (see Table 4.5).

TABLE 4.5

| | Rabies virus titer (log₁₀FFU/ml) | | | | | |
|---|---|---|---|---|---|---|
| | | Initial | | | | |
| | | yield | | | | |
| | PM and | after | 1 week at | | 2 weeks at | |
| Formul. | PMA* | drying | RT | 37° C. | RT | 37° C. |
| Non- | 7.86 ± 0.14 | 7.56 ± 0.12 | 7.68 ± 0.11 | 7.52 ± 0.11 | 7.50 ± 0.04 | 6.96 ± 0.27 |
| encaps. | | | | | | |
| Encaps. | 7.64 ± 0.02 | 5.88 ± 0.23 | 5.89 ± 0.26 | 5.33 ± 0.18 | 6.19 ± 0.07 | 4.94 ± 0.17 |

| | | | | |
|---|---|---|---|---|
| −80° C. | 7.70 ± 0.1 | 7.28 ± 0.21 | 7.25 ± 0.05 | 6.84 ± 0.12 |
| control | | | | |

*PM for non-encapsulated and PMA for encapsulated vaccine before drying.

Parallel titration of some specimens from these experiments was performed at CDC. They found that after one week at 37° C. activity was 5.26 log₁₀FFU/ml, which agrees with our measurements. They also measured activity after 1 hour equilibration at 65° C. It was equal to 5.7 log₁₀FFU/ml, which was close to initial vaccine activity after drying. Thus, equilibration at 65° C. did not significantly damage ERA-333 encapsulated in the gel microspheres. The damaging effect of storage at 37° C. was more pronounced. We suggested that that a reason of the losses in viral titer after preserving the vaccines in alginate gel microspheres is associated with the presence of high concentration of free Ca⁺⁺ ions. This suggestion was in agreement with the damaging effect on ERA-333 vaccine after 30 min equilibration in calcium chloride solutions with different concentration (see Table 4.6 below).

TABLE 4.6
Activity titer of ERA G333 Rabies vaccine after 30 min incubation
in culture media containing different concentrations of CaCl₂

| | |
|---|---|
| Concentration | Activity Titer (log₁₀FFU/ml 1) |

| of CaCl₂ (%) | RT | 0° C. |
|---|---|---|
| 1 | 6.94 ± 0.01 | 6.91 ± 0.01 |
| 0.5 | 7.26 ± 0.12 | 7.62 ± 0.001 |
| 0.1 | 7.4 ± 0.01 | 7.67 ± 0.03 |

Here it should be noted that in addition to the damaging effect of free Ca⁺⁺ there could be one more reason explaining why it is much more difficult to preserve vaccine encapsulated in gel microspheres. As we had elaborated in our original Phase I application, it is more difficult to remove water from the microspheres during secondary drying because the diffusion coefficient of water in strongly dehydrated microsphreres is also strongly decreased. For this reason, it could be damaging to increase the second temperature of stability drying to 45° C. if necessary (protective) level of dehydration was not achieved during previous steps of drying.

Thus, one of the possible approaches to increase survival of dry vaccines encapsulated in the gel microspheres could be to decrease the concentration of calcium chloride in the solutions used for microsphere preparation, Another approach could be based on decreasing the stability drying temperature. Both these approaches are in agreement with the results of the next experiment presented in the Table 4.7 below. In this experiment PMA was sprayed in CS solution containing only 0.1% of CaCl₂ instead 0.25% of CaCl₂ used in the pervious experiment. The results show that the damage is much smaller after stability drying at 40° C. than at 45° C.

TABLE 4.7
Effect of stability drying temperature on survival ERA-333
vaccine encapsulated in alginate gel microspheres.

| | ERA-333 virus titer (log₁₀FFU/ml) | | | | |
|---|---|---|---|---|---|
| | | Microspheres | | | |
| | | Before | After | 1 week at | |
| Formulation | PMA | drying | drying | RT | 37° C. |
| Microspheres | 7.75 ± 0.08 | 7.44 ± 0.2 | 6.64 ± 0.14 | 6.98 ± 0.1 | 5.99 ± 0.26 |
| dried at 40° C. | | | | | |
| Microspheres | | | 5.9 ± 0.32 | | |
| dried at 45° C. | | | | | |

| | | | |
|---|---|---|---|
| PM, −80° C. | 7.78 ± 0.2 | 7.48 ± 0.06 | 7.27 ± 0.12 |

Although the survival rate after drying increases with decreasing CaCl₂ concentration in CS, we found that it is difficult to obtain good firm gel particles using CS with concentration of calcium chloride below 0.1%. We also are concerned that further decrease in Ca⁺⁺ concentration could limit stability of the gel in the gastrointestinal (GI) tract and its protective role against gastric juice and bile. The solution that we had proposed was to use calcium D-gluconate instead of calcium chloride. Because calcium D-gluconate does not completely dissociate in water even at high concentration of calcium D-gluconate concentration of free Ca⁺⁺ (activity) could be small enough to avoid the damage that we observed using calcium chloride. The experimental observations below support this hypothesis.

4.2.1.3 Preservation of Vaccines Encapsulated in Alginate Gel Microsphere in Calcium D-gluconate Solutions.

We repeated the previous experiment with only one modification: we used 0.5% calcium D-gluconate instead of 0.1% CaCl₂ in the formulation of CS. The viral titer was tested at CDC after 2 week of storage at RT and 37° C. The results are shown in the table 4.8 below.

TABLE 4.8
Activity of ERA-333 vaccine encapsulated in alginate gel
microspheres in 0.5% calcium D-gluconate after drying and
subsequent 2 week storage at RT and 37° C.

| | ERA-333 virus titer after 2 weeks at | |
|---|---|---|
| Formulation | RT | 37° C. |
| Microspheres | 3.6. ± 1.2 \hat{ }6 (FFU/ml) | 9.3. ± 6.5 \hat{ }5 (FFU/ml) |
| dried at 40° C. | 6.56 (log₁₀FFU/ml) | 5.968 (log₁₀FFU/ml) |
| Microspheres | 3.8. ± 1.5 \hat{ }6 (FFU/ml) | 9.7. ± 4.17 \hat{ }5 (FFU/ml) |
| dried at 45° C. | 6.58 (log₁₀FFU/ml) | 5.986 (log₁₀FFU/ml) |

In this experiment titration was preformed at CDC. The original (frozen) virus titer was about 2.̂8 FFU/ml.

Surprisingly similar observations were observed for VRG, which is a more stable vaccine (see results presented in FIG. 2 (of two). In this experiment, to form PMA, VRG was mixed (1:5) with PS: 0.7% sodium alginate, 0.7% high amylase starch, 20% sucrose, 10% MAG, and 0.4% MSG. PMA was sprayed into two different CSs:
14% sucrose, 6% Mag, 0.2% MSG and 0.25% of CaCl₂; or
14% sucrose, 6% Mag, 0.2% MSG and 0.5% of calcium D-gluconate.

### DESCRIPTIION OF THE FIGURES

FIG. 1 (of two) demonstrates high survival and stability at 37° C. of PBV preserved VRG-AY 595-151. The preservation was performed using four different preservation solutions:
28% Isomalt and 12% MAG;
28% Sucrose and 12% MAG;
25.2% Sucrose, 10.8% MAG, and 4% MSG;
25.2% Isomalt, 10.8% Mannitol, and 4% MSG.

FIG. 2 (of two) demonstrates:
1. High survival and stability at 37° C. of PBV preserved VRG-AY 576-151 encapsulated in alginate gel if the microspheres were formed in 14% sucrose, 6% Mag, 0.2% MSG and 0.5% of calcium D-gluconate.
2. Low survival and stability at 37° C. of PBV preserved VRG-AY 576-151 encapsulated in alginate gel if the microspheres were formed in 14% sucrose, 6% Mag, 0.2% MSG and 0.25% of CaCl₂.

### REFERENCES

Blanton, J. D., Meadows, A., Murphy, S. M., Mnagan, J., Hanlon, C. A., Faber, M. L., Dietzschold, B. and Rupprecht, C. E. (2006) Vaccination of small Asian mongoose (Herpestes javanicus) against rabies. Journal of Wildlife Disease 42 (3), 663-666.
Bowersock, T. L., HogenEsch, H., Suckow, M., Guimond, P., Martin, S., Borie, D., Torregrosa, S., Park, H. and Park, K. (1999) Oral vaccination of animals with antigens encapsulated in alginate microspheres. Vaccine 17, 1804-1811.
Brochier, B., Kieny, M. P., Costy, F., Coppens, P., Bauduin, B., Lecocq, J. P., Languet, B., Chappuis, G., Desmettre, P., Afiademanyo, K., Libois, R. and Pstoret, P.-P. (1991) Large-scale eradication of rabies using recombinant vaccinia-rabies vaccine. pp. 520-522.
Bronshtein, V. (2004) Preservation by Foam Formulation: An Alternative to Freeze-Drying. Pharmaceutical Technology 28, 86-92.
Bronshtein, V. (1998) Preservation by Foam Formation, United States Pat. No. 5,766,520.
Bronshtein, V. (2004) Preservation by Foam Formulation: An Alternative to Freeze-Drying. Pharmaceutical Technology 28, 86-92.
Bronshtein, V. (2005) Preservation by Vaporization, PCT Patent Application WO051 1 7962A.
Bronshtein, V., Frank, J. L., and Leopold, A. C. (1996) Protection of desiccated enzymes by sugars. In: “Cryo 96 program”, Abstract 22 of a Paper Presented at the 33rd Annual Meeting of the Society for Cryobiology, Indianapolis, Ind.
Bronshtein, V., and Leopold, A. C. 1996. Accelerated aging of dried luciferase and isocitrate dehydrogenase. Effect of sugar/enzyme mass ratio. In: “Cryo 96 program”, Abstract 23 of a Paper Presented at the 33rd Annual Meeting of the Society for Cryobiology, Indianapolis, Ind.
Bronshtein, V. and Steponkus, P. L. (1993) Calorimetric Studies of Freeze-induced Dehydration of Phospholipids. Biophysical Journal 65, 1853-1865.
Bronshtein, V. L. and Chernov, A. A. (1991) Freezing potentials arising on solidification of dilute aqueous solution of electrolytes. Journal of Crystal Growth 112, 129-145.
Bryant, G. and Wolfe, J. (1992) Interfacial forces in cryobiology and anhydrobiology. Cryo-Letters 13, 23-36.
Burke, M. J. (1986) The glassy state and survival of anhydrous biological systems. In Membranes, Metabolism, and Dry Organisms ed. Leopold, A. C. pp. 358-363. Ithaca, N.Y.: Cornell University Press.
Calisher Charles H., James E. Childs, Hume E. Field, Kathryn V. Holmes, and Tony Schountz Bats (2006) Important Reservoir Hosts of Emerging Viruses Clinical Microbiology Reviews, 19, 531-545.
Chandramouli, V; Kailasapathy, K; Peiris, P; Jones, M (2004) An improved method of microencapsulation and its evaluation to protect Lactobacillus spp. in simulated gastric conditions. J. Microbiol. Methods. 2004 56(1), 27-35.
Dietzschold, B., Faber, M. and Schnell, J. (2003) New approaches to the prevention and eradication of rabies. Expert Rev. Vaccines 2, 399-406.
Franks, F. (1990) Freeze drying: From empiricism to predictability. Cryo-Letters 11, 93-100.
Franks, F. (1991) Freeze-drying: from empiricism to predictability. The significance of glass transitions. Dev. Biol. Stand. 74, 9-19.
Franks, F. (1994) Long-term stabilization of biologicals. Bio/Technology 12, 253-256.
Israelachvili, J. N. (1985) Measurements of hydration forces between macroscopic surfaces. Chemica Scripta 25, 7-14.
Jojola, S. M., Robinson, S. J. and VerCauteren, K. C. (2004) Oral rabies vaccine (ORV) bait uptake by captive striped skunks. Journal of Wildlife Diseases 43, 97-106.
Krauss Hartmut et al. (2003) Zoonoses: infectious diseases transmissible from animals to humans, ASM Press, 3rd ed.
Lee Ki-Yong and Tae-Ryeon Heo (2000) Survival of Bifidobacterium longum Immobilized in Calcium Alginate Beads in Simulated Gastric Juices and Bile Salt Solution. Applied and Environmental Microbiology 66, 869-873.
Mandal S., A. K. Puniyaa and K. Singha (2006) Effect of alginate concentrations on survival of microencapsulated Lactobacillus casei NCDC-298. International Dairy Journal 16, 1190-1195.
Marcelja, S. (1982) Molecular mechanism of the hydration force. In Biophysics of Water ed. Franks, F. and Mathias, S. pp. 176-177. Chichester: Wiley and Sons.
Maurice, S., Nussinovitch, A., Jaffe, N., Shoseyov, O. and Gertler, A. (2004) Oral immunization of Carassius auratus with modified recombinant A-layer proteins entrapped in alginate beads. Vaccine 23, 450-459.
Meslin, F.-X., Fishbein, D. B. and Matter, H. C. (1994) Rationale and Prospects for Rabies Elimination in Developing Countries. Curr Top Microbiol Immunol 187, 1-26.
Orciari, L. A., Niezgoda, M., Hanlon, C. A., Shaddock, J. H., Sanderlin, D. W., Yager, P. A. and Rupprecht, C. E. (2001) Rapid clearance of SAG-2 rabies virus from dogs after oral vaccination. Vaccine 19, 4511-4518.
Parsegian, V. A. (1973) Long-range physical forces in the biological mileau. Annual Review of Biophysics and Bioengineering 2, 221-255.
Pincet, F., Perez, E. and Wolfe, J. (1994) Do trehalose and dimethyl sulfoxide affect intermembrane forces? Cryobiology 31, 531-539.
Rupprecht, C. E. (2006) Current and future trends in the prevention, treatment and control of rabies. pp. 1021-1038.
Rupprecht, C. E., Blass, L., Smith, K., Orciari, L. A., Niezgoda, M., Whitfield, S. G., Gibbons, R. V., Guerra, M. and Hanlon, C. A. (2001) Human infection due to recombinant vaccinia-rabies glycoprotein virus. N Engl J Med 345, 582-586.
Rupprecht, C. E., Hanlon, C. A., Blanton, J., Manangan, J., Morrill, P., Murphy, S., Niezgoda, M., Orciari, L. A., Schumacher, C. L. and Dietzschold, B. (2005) Oral vaccination of dogs with recombinant rabies virus vaccines. pp. 101-105.
Sailaja G., H. HogenEsch, A. North, J. Hays, and S. K. Mittal (2002) Encapsulation of Recombinant Adenovirus into Alginate Microspheres Circumvents Vector Specific Immune Response. Gene Ther 2002 9(24), 1722-1729.
Seo Jun-Young, Seung Yong Seong, Byung-Yoon Ahn, Ick Chan Kwon, Hesson Chung, and Seo Young Jeong (2002) Cross-Protective Immunity of Mice Induced by Oral Immunization with Pneumococcal Surface Adhesin A Encapsulated in Microspheres Infect Immun. 70 (3), 1143-1149.
Sultana Khalida, Georgia Godwarda, N. Reynoldsb, R. Arumugaswamyc, P. Peirisc and Kaila Kailasapathy (2000) Encapsulation of probiotic bacteria with alginate-starch and evaluation of survival in simulated gastrointestinal conditions and in yoghurt. International Journal of Food Microbiology 62, 47-55.
Slate, D., Rupprecht, C. E., Rooney, J. A., Donovan, D., Lein, D. H. and Chipman, R. B. (2005) Status of oral rabies vaccination in wild carnivores in the United States. Virus Research 111, 68-76.
Winkler, W. G. and Bogel, K. (1992) Control of Rabies in Wildlife. Sci Am 266, 86-92.

What is claimed is:

1. The method of long-term stabilization of biologics at high ambient temperatures (room temperature and above) in which, after a primary drying, one performs a secondary drying, comprising at least two steps of stability drying at elevated temperatures (above the room temperature: 35° C., 40° C., 45° C., 50° C., and higher temperatures) and subsequent cooling to the storage temperature to achieve vitrification.

2. The method, according to 1, wherein to protect from desiccation stress, biologics are placed in preservation solutions comprising isomalt and methylglucoside.

3. Cryo-encapsulating procedure for encapsulating biologics in alginate gel microspheres comprising the following steps:
a) Spraying of a preservation mixture (PM) comprising the biologics and alginate into liquid nitrogen (LN₂) (Cryopelletization) using a sprayer, which produces frozen particles with diameters of about 100-300μ.
b) Spraying a crosslinking solution containing a source Ca⁺⁺ into the same tray with LN₂.
c) Mixing the frozen microspheres in LN₂ and separating the microspheres from the LN₂.
d) Warming the frozen mixture of microspheres to obtain a concentrated suspension alginate gel microspheres.

4. The procedure according to 3 wherein the source Ca⁺⁺ is calcium gluconate.
